(19) United States
(12) Patent Application Publication
(10) Pub. No.: US 2025/0278829 A1
(43) Pub. Date: Sep. 4, 2025

(54) METHOD AND DEVICE FOR PROCESSING THREE-DIMENSIONAL ORAL CAVITY MODEL

(71) Applicant: MEDIT CORP., Seoul (KR)

(72) Inventor: Haram JEON, Seoul (KR)

(73) Assignee: MEDIT CORP., Seoul (KR)

(21) Appl. No.: 18/580,414

(22) PCT Filed: Jul. 21, 2021
(86) PCT No.: PCT/KR2022/010717

§ 371 Date: Aug. 7, 2024

(30) Foreign Application Priority Data
Jul. 21, 2021 (KR) 10-2021-0095623
Nov. 10, 2021 (KR) 10-2021-0154052

(51) Int. Cl. G06T 7/00; A61C 9/00; G06T 17/00; G16H 50/50

(52) U.S. Cl. CPC G06T 7/0012; A61C 9/0053; G06T 17/00; G16H 50/50; G06T 2207/30036

(57) ABSTRACT

Provided are a method and apparatus for processing a three-dimensional intraoral model. The method of processing a three-dimensional intraoral model includes obtaining scan data by scanning an object, generating a border of a base based on the scan data, obtaining a gingiva area from the scan data, and obtaining tooth model data by generating a mesh between the gingiva area and the border of the base and connecting the gingiva area and the border of the base by using the mesh, another mesh different from the mesh being generated at a position corresponding to a palate.

### TECHNICAL FIELD

Described embodiments relate to a method and apparatus for processing a three-dimensional intraoral (or oral cavity) model. In particular, described embodiments relate to a method and apparatus for processing a three-dimensional intraoral model used to generate a natural tooth model.

### BACKGROUND ART

There are various fields in the dental treatment of patients. Examples of the dental treatment may include an orthodontic treatment and a prosthetic treatment. A model of the patient's teeth may be necessary for an orthodontic treatment and a prosthetic treatment. A tooth model may be objective data that shows the appearances before, during, and after an orthodontic treatment. Also, by making a tooth model, it may be easy to directly observe a portion that is difficult to directly observe due to a restricted space in an oral cavity. Also, it may be possible to accurately measure a space that is required to eliminate a misaligned tooth, a projecting tooth, a void, or the like. Also, when a doctor consults with a patient, a tooth model may serve as objective data for showing the patient's intraoral condition to help the patient's understanding. Also, a device that is difficult to directly make in the patient's oral cavity may be modeled.

When a patient's tooth model is generated based on scan data of the patient's oral cavity, a method of processing a suitable three-dimensional intraoral model by using the scan data is required in order to generate a more natural tooth model.

### DISCLOSURE

### Technical Problem

Described embodiments relate to a method and apparatus for processing a three-dimensional intraoral model for generating a natural tooth model.

### Technical Solution

According to an embodiment, a method of processing a three-dimensional intraoral model includes obtaining scan data by scanning an object, generating a border of a base based on the scan data, obtaining a gingiva area from the scan data, and obtaining tooth model data by generating a mesh between the gingiva area and the border of the base and connecting the gingiva area and the border of the base by using the mesh, another mesh different from the mesh being generated at a position corresponding to a palate.

According to an embodiment, the obtaining of the tooth model data may include generating the another mesh at the position corresponding to the palate by using an auxiliary line.

According to an embodiment, the obtaining of the tooth model data may include generating the another mesh by generating the auxiliary line at the position corresponding to the palate in the gingiva area and extending boundary points of the gingiva area corresponding to the palate to the auxiliary line.

According to an embodiment, the obtaining of the tooth model data may include mapping the boundary points of the gingiva area corresponding to the palate to points of the auxiliary line and generating a mesh based on the mapped points, and generating a mesh inside the auxiliary line.

According to an embodiment, the auxiliary line may include a curve or a parabola.

According to an embodiment, the obtaining of the tooth model data may include dividing a boundary of the gingiva area into a plurality of boundary zones to correspond to sides of a polygon constituting the base, generating points at a side of the base corresponding to each boundary zone to correspond to points of each boundary zone of the gingiva area, and generating the another mesh by mapping the points of each boundary zone of the gingiva area to the points of the side corresponding to each boundary zone.

According to an embodiment, the obtaining of the tooth model data may include, with respect to a zone corresponding to the palate, mapping at least some of points of a boundary zone of the gingiva area to points of the auxiliary line and generating a mesh based on the mapped points, and generating a mesh inside the auxiliary line.

According to an embodiment, the obtaining of the gingiva area may include identifying a tooth portion and a gingiva portion from the scan data and obtaining the gingiva area by generating a gingiva sidewall by extending the gingiva portion.

According to an embodiment, a data processing apparatus for processing a three-dimensional intraoral model includes a memory storing one or more instructions, and a processor configured to execute the one or more instructions stored in the memory to obtain scan data by scanning an object, generate a border of a base based on the scan data, obtain a gingiva area from the scan data, and obtain tooth model data by generating a mesh between the gingiva area and the border of the base and connecting the gingiva area and the border of the base by using the mesh, another mesh different from the mesh being generated at a position corresponding to a palate.

According to an embodiment, a computer-readable recording medium has recorded thereon a program for performing, by a data processing apparatus, a method of processing a three-dimensional intraoral model, the method including obtaining scan data by scanning an oral cavity, generating a border of a base based on the scan data, obtaining a gingiva area from the scan data, and obtaining tooth model data by generating a mesh between the gingiva area and the border of the base and connecting the gingiva area and the border of the base by using the mesh, another mesh different from the mesh being generated at a position corresponding to a palate.

### Advantageous Effects

According to the method and apparatus for processing a three-dimensional intraoral model according to the described embodiments, a more natural tooth model may be generated.

### DESCRIPTION OF DRAWINGS

The present disclosure may be easily understood through the following detailed description and the accompanying drawings, in which reference numerals refer to structural elements.

FIG. 1 is a diagram for describing a digital intraoral model processing system according to described embodiments.

FIG. 2 is a reference diagram for describing tooth model data with a base combined therewith according to an example.

FIG. 3 is a block diagram illustrating a data processing apparatus according to described embodiments.

FIG. 4 is a flowchart illustrating a method of processing a three-dimensional intraoral model in a data processing apparatus, according to described embodiments.

FIG. 5 illustrates an example of a three-dimensional intraoral model obtained by the data processing apparatus, according to an example.

FIG. 6 is a reference diagram for describing an example of manually receiving an occlusion axis of scan data in the data processing apparatus, according to an embodiment.

FIG. 7 is a reference diagram for describing an example of a method of generating a border of an upper-jaw base, according to an embodiment.

FIG. 8 is a reference diagram for describing a method of obtaining a gingiva area from scan data.

FIG. 9 is a reference diagram for describing a method of dividing an area between the gingiva area and the base into a plurality of zones and generating a mesh in an outer area, according to an embodiment.

FIG. 10 is a reference diagram for describing a method of generating a curve in an area corresponding to the position of a palate, according to an embodiment.

FIG. 11 is a reference diagram for describing a method of dividing an area between the gingiva area and the base into a plurality of zones and generating a mesh in an inner area, according to an embodiment.

FIG. 12 is a reference diagram for describing a method of generating a mesh for each subarea in a seventh zone that is an inner area, according to an embodiment.

### MODE FOR INVENTION

The specification clarifies the scope of the present disclosure and describes the principle of the present disclosure and embodiments so that those of ordinary skill in the art may implement the present disclosure. The described embodiments may be implemented in various forms.

Throughout the specification, like reference numerals will denote like elements. The specification may not describe all elements of the embodiments, and general descriptions in the art to which the present disclosure belongs or redundant descriptions between the embodiments will be omitted for conciseness. The term ‘unit’ (or part or portion) used herein may be implemented as software or hardware, and depending on the embodiments, a plurality of ‘units’ may be implemented as one element (or unit) or one ‘unit’ may include a plurality of elements. Hereinafter, the operation principle and embodiments of the present disclosure will be described with reference to the accompanying drawings.

Herein, an image may include an image representing at least one tooth or an oral cavity including at least one tooth (hereinafter referred to as “intraoral image”).

Also, herein, the image may be a two-dimensional image of an object or a three-dimensional (3D) model or a three-dimensional image stereoscopically representing an object. Also, herein, the image may refer to data required to two-dimensionally or three-dimensionally represent an object, for example, raw data obtained from at least one image sensor. Particularly, the raw data may be data obtained to generate an intraoral image, and when the inside of the oral cavity of a patient as an object is scanned by using an intraoral scanner, the raw data may be data (e.g., two-dimensional data) obtained from at least one image sensor included in the intraoral scanner.

Herein, the ‘object’ may include a tooth, a gingiva, at least some area of the oral cavity, and/or an artificial structure insertable into the oral cavity (e.g., an orthodontic device, an implant, an artificial tooth, or an orthodontic assistance tool inserted into the oral cavity). Here, the orthodontic device may include at least one of a bracket, an attachment, an orthodontic screw, a lingual orthodontic device, and a removable orthodontic retainer.

Hereinafter, embodiments will be described in detail with reference to the accompanying drawings.

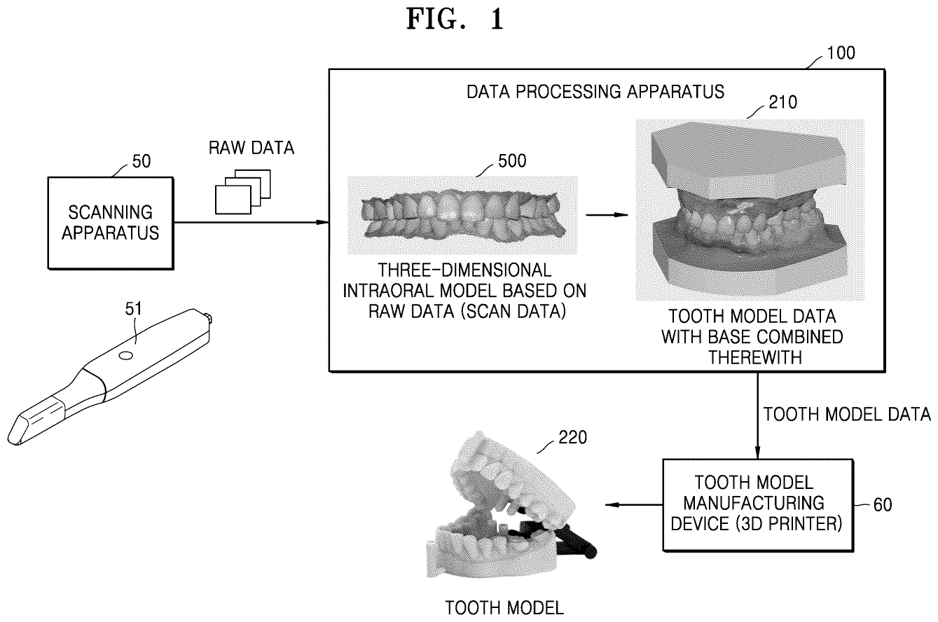

FIG. 1 is a diagram for describing a digital intraoral model processing system according to described embodiments.

Referring to FIG. 1, the digital intraoral model processing system may include a scanning device 50 and a data processing apparatus 100.

The scanning device 50 may be an apparatus for scanning an object, and the object may include any object or body that is to be scanned. For example, the object may include at least a portion of the patient's body including an oral cavity (mouth) or a face, or a tooth model. The scanning device may include a handheld scanner that the user holds in his/her hand to scan an object, or a model scanner that installs a tooth model and scans an object while moving around the installed tooth model.

For example, as an example of the handheld scanner, an intraoral scanner 51 may be a device for obtaining an image of the oral cavity including at least one tooth by being inserted into the oral cavity and scanning teeth in a non-contact manner. Also, the intraoral scanner 51 may have a form capable of being inserted into and withdrawn from the oral cavity and may scan the inside of the patient's oral cavity by using at least one image sensor (e.g., an optical camera). In order to image the surface of at least one of a tooth and a gingiva inside the oral cavity and an artificial structure insertable into the oral cavity (e.g., an orthodontic device including brackets and wires, an implant, an artificial tooth, or an orthodontic assistance tool inserted into the oral cavity) which is an object, the intraoral scanner 51 may obtain surface information about the object as raw data. The intraoral scanner 51 may be suitable for scanning the oral cavity by being designed to be easily inserted into and withdrawn from the oral cavity; however, a body region such as the patient's face or the like may also be scanned by using the intraoral scanner 51.

The scanning device 50 may obtain image data by using an optical triangulation method, a confocal method, or other methods.

The image data obtained by the scanning device 50 may be transmitted to the data processing apparatus 100 connected through a wired or wireless communication network.

The data processing apparatus 100 may be any electronic apparatus that may be connected to the scanning device 50 through a wired or wireless communication network, may receive a two-dimensional image obtained by scanning the oral cavity from the scanning device 50, and may generate, process, display, and/or transmit an intraoral image based on the received two-dimensional image.

Based on the two-dimensional image data received from the scanning device 50, the data processing apparatus 100 may generate at least one of information generated by processing the two-dimensional image data and an intraoral image generated by processing the two-dimensional image data and may display the generated information and intraoral image through a display.

The data processing apparatus 100 may be, but is not limited to, a computing device such as a smart phone, a laptop computer, a desktop computer, a PDA, or a tablet PC.

Also, the data processing apparatus 100 may be provided in the form of a server (or a server device) for processing an intraoral image.

Also, the scanning device 50 may intactly transmit, to the data processing apparatus 100, the raw data obtained through scanning. In this case, based on the received raw data, the data processing apparatus 100 may generate a three-dimensional intraoral image three-dimensionally representing the oral cavity. Also, because the ‘three-dimensional intraoral image’ may be generated by three-dimensional modeling of the internal structure of the oral cavity based on the received raw data, it may also be referred to as a ‘three-dimensional intraoral model’, a ‘digital intraoral model’, or a ‘three-dimensional intraoral image’. Hereinafter, a model or an image two-dimensionally or three-dimensionally representing the oral cavity will be collectively referred to as an ‘intraoral image’.

Also, the data processing apparatus 100 may analyze, process, display, and/or transmit the generated intraoral image to an external device.

As another example, the scanning device 50 may obtain raw data through scanning, generate an image corresponding to the oral cavity as an object by processing the obtained raw data, and transmit the image to the data processing apparatus 100. In this case, the data processing apparatus 100 may analyze, process, display, and/or transmit the received image.

In embodiments, the data processing apparatus 100 may be an electronic device that may generate and display an intraoral image three-dimensionally representing the oral cavity including one or more teeth, which will be described below in detail.

According to an embodiment, when the data processing apparatus 100 receives raw data of obtained by scanning the oral cavity from the scanning device 50, the data processing apparatus 100 may process the received raw data to generate a three-dimensional intraoral model. The raw data received from the scanning device 50 may include a tooth portion representing teeth and a gingiva portion representing gingivae. Thus, the three-dimensional intraoral model generated by the data processing apparatus 100 may include a tooth portion representing teeth and a gingiva portion representing gingivae.

According to an embodiment, the data processing apparatus 100 may generate tooth model data with a base combined therewith such that it may be used to generate a tooth model based on an initial three-dimensional intraoral model generated based on the raw data. In the following description, for convenience, the three-dimensional intraoral model generated based on the raw data will be referred to as scan data.

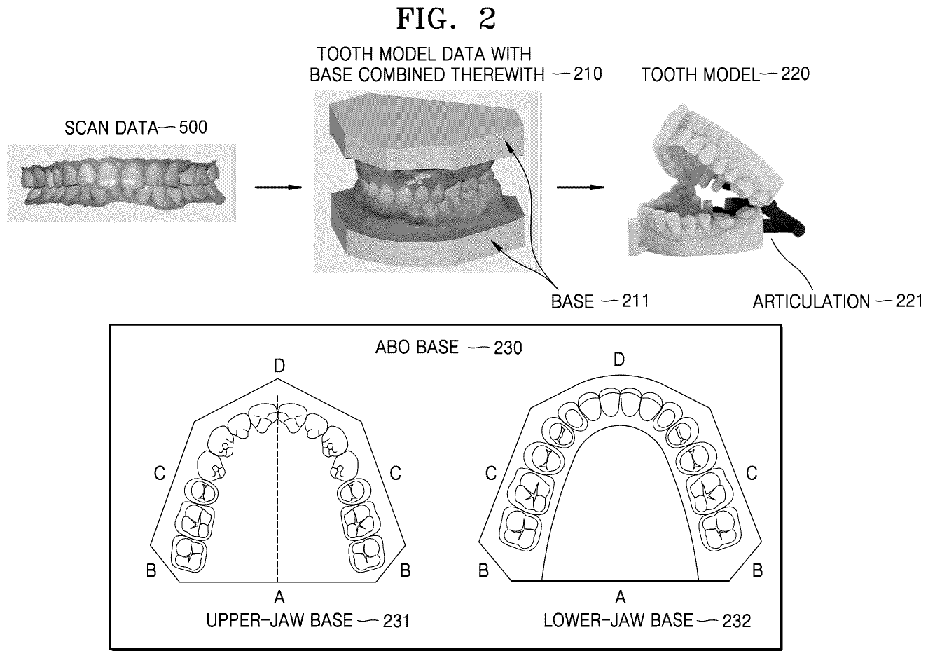

FIG. 2 is a reference diagram for describing tooth model data with a base combined therewith according to an example.

Referring to FIG. 2, when a tooth model is generated by using only scan data 500 obtained by scanning the patient's oral cavity, the upper jaw and the lower jaw included in the scan data are separated as they are and thus it may be impossible to know accurate occlusion of the teeth. Thus, as illustrated in FIG. 2, in order to generate a tooth model in a form in which an articulation 221 is attachable to a tooth model 220, it may be desirable to generate a tooth model by combining a base 211 with tooth model data 210 used to generate a tooth model.

A base for combining an upper-jaw (maxillary) structure with a low-jaw (mandibular) structure may be in various forms. For example, there may be a plate base type in which a plate is formed at a palate region and a tongue portion, and a plateless base type in which a palate region and a tongue portion are opened.

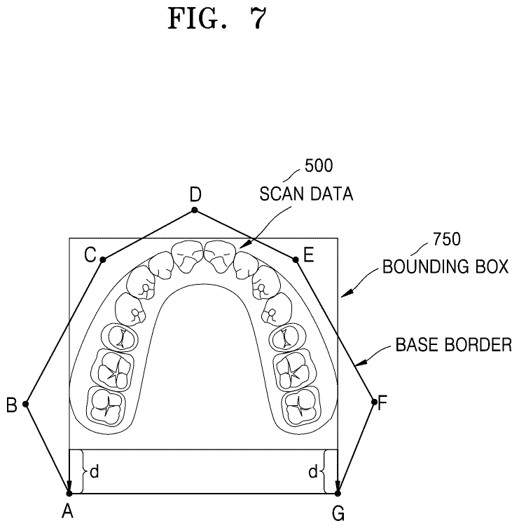

Also, there may be an ABO base type 230 according to the American Board of Orthodontics standards. Referring to FIG. 2, the ABO base type may include a lower-jaw base 232 and an upper-jaw base 231 including a palate region, wherein the upper-jaw base may be heptagonal and the lower-jaw base may include an elliptical front-tooth portion. Embodiments described with reference to FIGS. 7 to 11 will be described by using this ABO base type as an example.

Returning back to FIG. 1, according to an embodiment, the data processing apparatus 100 may generate a border of a base based on scan data and obtain tooth model data 210 by generating a mesh between a gingiva area of the scan data and the border of the base. In this case, the data processing apparatus 100 may obtain tooth model data by generating another mesh different from the above mesh at a position corresponding to the palate.

According to an embodiment, the data processing apparatus 100 may generate the another mesh by using an auxiliary line at the position corresponding to the palate.

According to an embodiment, the data processing apparatus 100 may generate the another mesh by generating an auxiliary line at the position corresponding to the palate in the gingiva area and extending a boundary point of the gingiva area corresponding to the palate to the auxiliary line.

According to an embodiment, when generating a mesh, the data processing apparatus 100 may map a boundary point of the gingiva area corresponding to the palate to a point of the auxiliary line, generate a mesh based on the mapped points, and generate a mesh inside the auxiliary line.

According to an embodiment, the auxiliary line may include a curve or a parabola.

According to an embodiment, the data processing apparatus 100 may divide a boundary of the gingiva area into a plurality of boundary zones to correspond to sides of a polygon constituting the base, generate points at a side of the base corresponding to each boundary zone to correspond to points of each boundary zone of the gingiva area, and generate another mesh by mapping the points of each boundary zone of the gingiva area to the points of the side corresponding to each boundary zone.

When the tooth model data generated by the data processing apparatus 100 is provided to a tooth model manufacturing device 60 such as a 3D printer, the tooth model manufacturing device 60 may generate the tooth model 220 based on the tooth model data 210.

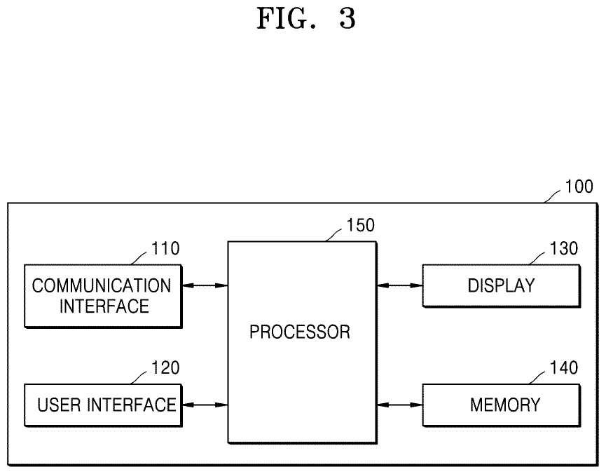

FIG. 3 is a block diagram illustrating a data processing apparatus 100 according to described embodiments.

Referring to FIG. 3, the data processing apparatus 100 may include a communication interface 110, a user interface 120, a display 130, a memory 140, and a processor 150.

The communication interface 110 may communicate with at least one external electronic device through a wired or wireless communication network. Particularly, the communication interface 110 may communicate with the scanning apparatus 50 under the control by the processor 150. Under the control by the processor 150, the communication interface 110 may communicate with an external electronic device or server or the like connected through a wired or wireless communication network.

The communication interface 110 may communicate with an external electronic device (e.g., an intraoral scanner, a server, or an external medical device) through a wired or wireless communication network. Particularly, the communication interface may include at least one short-range communication module performing communication according to the communication standard such as Bluetooth, Wi-Fi, Bluetooth Low Energy (BLE), NFC/RFID, Wi-Fi Direct, UWB, or ZigBee.

Also, the communication interface 110 may further include a long-distance communication module for performing communication with a server to support long-distance communication according to the long-range communication standard. Particularly, the communication interface 110 may include a long-range communication module performing communication through a network for Internet communication. Also, the communication interface may include a long-range communication module performing communication through a communication network conforming to the communication standard such as 3G, 4G, and/or 5G.

Also, in order to communicate by wire with an external electronic device (e.g., an intraoral scanner), the communication interface 110 may include at least one port for being connected to the external electronic device through a wired cable. Accordingly, the communication interface 110 may perform communication with the external electronic device connected by wire through the at least one port.

The user interface 120 may receive a user input for controlling the data processing apparatus 100. The user interface 120 may include, but is not limited to, a user input device including a touch panel for sensing a user's touch, a button for receiving a user's push operation, and/or a mouse or a keyboard for designating or selecting a point on a user interface screen.

Also, the user interface 120 may include a voice recognition device for voice recognition. For example, the voice recognition device may be a microphone, and the voice recognition device may receive a user's voice command or voice request. Accordingly, the processor may control an operation corresponding to the voice command or voice request to be performed.

The display 130 may display a screen. Particularly, the display 130 may display a certain screen under the control by the processor 150. Particularly, the display 130 may display a user interface screen including the intraoral image generated based on the data obtained by scanning the patient's oral cavity by the scanning apparatus 50. Alternatively, the display 130 may display a user interface screen including information related to the patient's dental treatment.

The memory 140 may store at least one instruction. Also, the memory 140 may store at least one instruction executed by the processor 150. Also, the memory may store at least one program executed by the processor 150. Also, the memory 140 may store raw data received from an intraoral scanner, or an intraoral image three-dimensionally representing the oral cavity.

According to an embodiment, the memory 140 may store a program for generating a three-dimensional intraoral model by processing the raw data. As such, a program for generating a three-dimensional intraoral model by processing the raw data may be downloaded from a server computer and then stored therein.

According to an embodiment, the memory 140 may store a program for generating tooth model data based on a three-dimensional intraoral model according to the method described herein. As such, a program for generating tooth model data may be downloaded from a server computer and then stored therein.

The processor 150 may execute at least one instruction stored in the memory 140 to perform control such that a desired operation may be performed. Here, the at least one instruction may be stored in an internal memory included in the processor 150 or in the memory 140 included in the data processing apparatus separately from the processor.

Particularly, the processor 150 may execute at least one instruction to control at least one component included in the data processing apparatus such that a desired operation may be performed. Thus, although a case where the processor performs certain operations is described as an example, it may mean that the processor controls at least one component included in the data processing apparatus such that certain operations may be performed.

According to an embodiment, by executing one or more instructions stored in the memory 140, the processor 150 may obtain scan data by scanning an object, generate a border of a base based on the scan data, obtain a gingiva area from the scan data, and obtain tooth model data by generating and connecting a mesh between a boundary point of the gingiva area and the border of the base and generating a mesh at a position corresponding to the palate by using an auxiliary line.

According to an embodiment, by executing one or more instructions stored in the memory 140, the processor 150 may generate an auxiliary line at a position corresponding to the palate in the gingiva area and extend a boundary point of the gingiva area corresponding to the palate to the auxiliary line to generate a mesh.

According to an embodiment, by executing one or more instructions stored in the memory 140, the processor 150 may map a boundary point of the gingiva area corresponding to the palate to a point of the auxiliary line, generate a mesh based on the mapped points, and generate a mesh inside the auxiliary line. According to an embodiment, by executing one or more instructions stored in the memory 140, the processor 150 may divide a boundary of the gingiva area into a plurality of zones to correspond to sides of a polygon constituting the base, generate points at a side of the base corresponding to each boundary zone to correspond to points of each boundary zone of the gingiva area, and generate a mesh by mapping the points of each boundary zone of the gingiva area to the points of the side corresponding to each boundary zone. According to an embodiment, by executing one or more instructions stored in the memory 140, the processor 150 may identify a tooth portion and a gingiva portion from the scan data and obtain a gingiva area by generating a gingiva sidewall by extending the gingiva portion.

According to an example, the processor 150 may internally include at least one internal processor and a memory device (e.g., RAM or ROM) for storing at least one of programs, instructions, signals, and data to be processed or used by the internal processor.

Also, the processor 150 may include a graphic processor (graphic processing unit) for graphic processing corresponding to video. Also, the processor may be implemented as a System-on-Chip (SoC) including a combination of a core and a GPU. Also, the processor may include a single core or a multi-core. For example, the processor may include a dual core, a triple core, a quad core, a hexa core, an octa core, a deca core, a dodeca core, a hexadecimal core, and/or the like.

In the described embodiments, the processor 150 may generate an intraoral image based on the two-dimensional image received from the scanning device 50.

Particularly, under the control by the processor 150, the communication interface 110 may receive the data obtained by the scanning device 50, for example, the raw data obtained through intraoral scanning. Based on the raw data received by the communication interface, the processor 150 may generate a three-dimensional intraoral image three-dimensionally representing the oral cavity. For example, in order to restore a three-dimensional image according to an optical triangulation method, the intraoral scanner may include at least one camera and may include an L camera corresponding to the left field of view and an R camera corresponding to the right field of view in a particular embodiment. The intraoral scanner may obtain L image data corresponding to the left field of view and R image data corresponding to the right field of view from the L camera and the R camera, respectively. Subsequently, the intraoral scanner (not illustrated) may transmit raw data including the L image data and the R image data to the communication interface of the data processing apparatus 100.

Then, the communication interface 110 may transmit the received raw data to the processor, and the processor may generate an intraoral image three-dimensionally representing the oral cavity based on the received raw data.

Also, the processor 150 may control the communication interface to directly receive an intraoral image three-dimensionally representing the oral cavity from an external server, a medical device, or the like. In this case, the processor may obtain a three-dimensional intraoral image without generating a three-dimensional intraoral image based on the raw data.

According to embodiments, the processor 150 performing operations such as “extracting”, “obtaining”, and “generating” may include not only the processor 150 executing at least one instruction to directly perform the above operations but also the processor 150 controlling other components to perform the above operations.

In order to implement the embodiments described herein, the data processing apparatus 100 may include only some of the components illustrated in FIG. 3 or may include more components than the components illustrated in FIG. 3.

Also, the data processing apparatus 100 may store and execute dedicated software linked to the intraoral scanner. Here, the dedicated software may also be referred to as a dedicated program, a dedicated tool, or a dedicated application. When the data processing apparatus 100 operates in conjunction with the scanning device 50, the dedicated software stored in the data processing apparatus 100 may be connected to the scanning device 50 to receive the data obtained through intraoral scanning in real time. For example, there is dedicated software for processing data obtained through intraoral scanning by an intraoral scanner product of Medit. Particularly, Medit produces and distributes software for processing, managing, using, and/or transmitting data obtained by the intraoral scanner. Here, because the “dedicated software” refers to a program, a tool, or an application operable in conjunction with the intraoral scanner, various intraoral scanners developed and sold by various manufacturers may be used in common. Also, the above dedicated software may be produced and distributed separately from the intraoral scanner for performing intraoral scanning.

The data processing apparatus 100 may download, from a server computer, dedicated software for processing data obtained through intraoral scanning by the Medit's intraoral scanner product and store and execute the same. The dedicated software may perform one or more operations for obtaining, processing, storing, and/or transmitting the intraoral image. Here, the dedicated software may be stored in the processor. Also, the dedicated software may provide a user interface for using the data obtained by the intraoral scanner. Here, the user interface screen provided by the dedicated software may include the intraoral image generated according to the described embodiments.

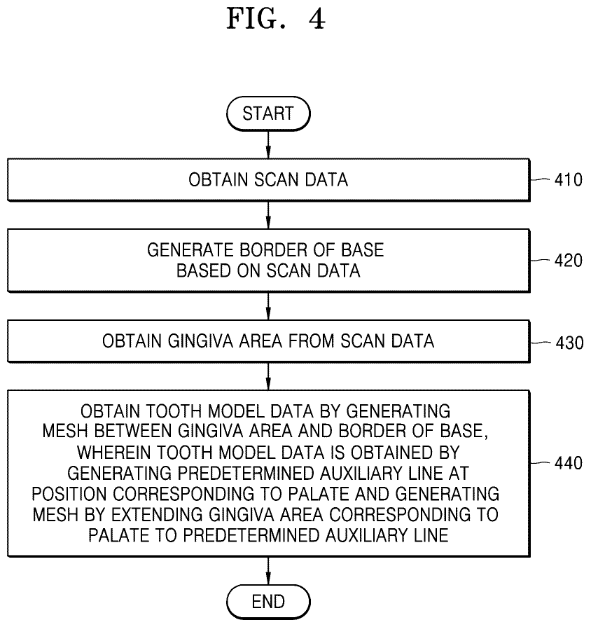

FIG. 4 is a flowchart illustrating a method of processing a three-dimensional intraoral model in a data processing apparatus, according to described embodiments. The three-dimensional intraoral model processing method illustrated in FIG. 4 may be performed through the data processing apparatus 100.

Referring to FIG. 4, in operation 410, the data processing apparatus 100 may obtain scan data by scanning the oral cavity.

The data processing apparatus 100 may receive raw data obtained by scanning the patient's oral cavity or scanning a tooth model from the scanning device 50 and obtain a three-dimensional intraoral model including a tooth portion and a gingiva portion by processing the received raw data. Alternatively, the data processing apparatus 100 may obtain raw data or a three-dimensional intraoral model stored in the memory.

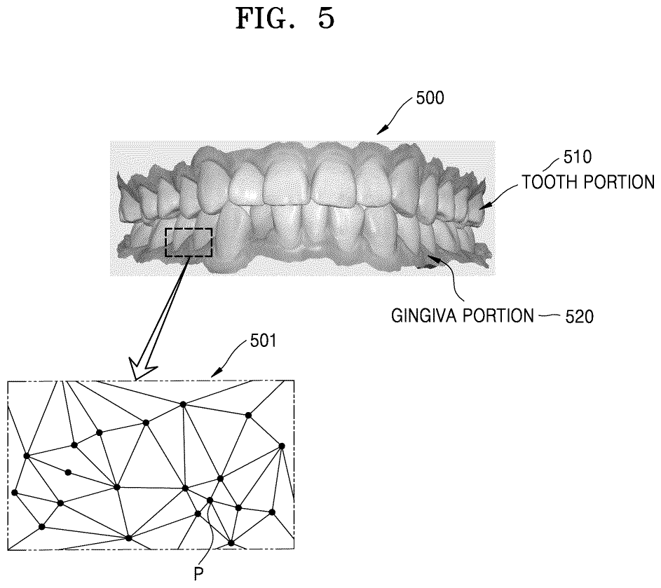

FIG. 5 illustrates an example of a three-dimensional intraoral model obtained by the data processing apparatus 100, according to an example.

For example, when two-dimensional data is obtained by using an intraoral scanner, the data processing apparatus 100 may calculate the coordinates of a plurality of illuminated surface points by using a triangulation method. By using an intraoral scanner to perform scanning while moving along the surface of an object, the coordinates of the surface points may be accumulated as the amount of scan data increases. As a result of the image obtainment, a point cloud of vertexes may be identified to represent the extent of the surface. The points in the point cloud may represent actually measured points on the three-dimensional surface of the object. The surface structure may be approximated by forming a polygonal mesh in which adjacent vertexes of the point cloud are connected by a line segment. The polygonal mesh may be variously determined, such as a triangular mesh, a square mesh, or a pentagonal mesh. The relationships between the polygon of a mesh model and an adjacent polygon may be used to extract features of the tooth boundary, such as curvature, minimum curvature, edge, and spatial relationship.

Referring to FIG. 5, an area 501 of a three-dimensional intraoral model 500 may include a plurality of vertexes constituting a point cloud and a triangular mesh generated by connecting adjacent vertexes by lines.

Referring to FIG. 5, the three-dimensional intraoral model 500 may include a tooth portion 510 and a gingiva portion 520. In the case of the tooth portion 510, because the tooth protrudes from the gingiva, the shape of the tooth exposed to the outside may be obtained by scanning while moving around the tooth by using the scanning device 50. In the case of the gingiva portion 520, because the gingiva is a portion between the tooth and another mucosa in the oral cavity, the boundary between the gingiva and the mucosa is unclear, and particularly, the edge portion thereof is a portion connected to other mucosa in the oral cavity, it may be difficult to scan and obtain all the cross-sections thereof. Thus, it is illustrated that the representation of the gingiva portion 520 is not smooth. Hereinafter, for convenience, the three-dimensional intraoral model will be referred to as scan data.

Next, the data processing apparatus 100 may align an occlusion axis of the scan data obtained as such.

According to an embodiment, the data processing apparatus 100 may automatically align the occlusion axis of the scan data.

According to an embodiment, the data processing apparatus 100 may manually receive the occlusion axis of the scan data through a user input.

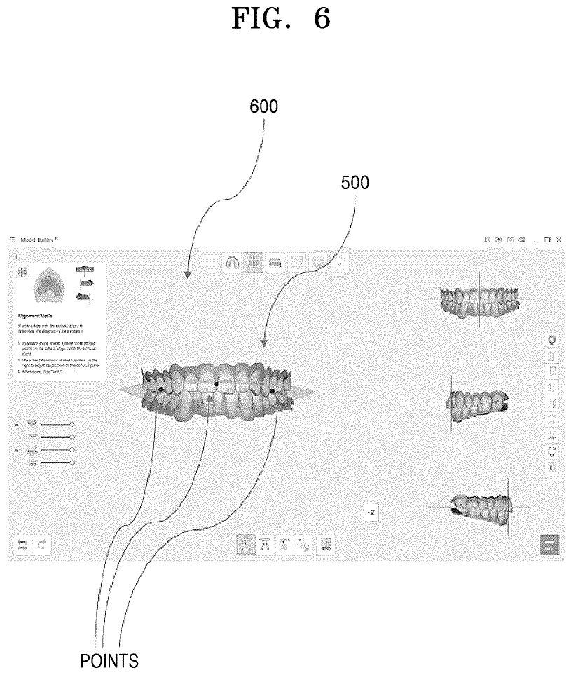

FIG. 6 is a reference diagram for describing an example of manually receiving an occlusion axis of scan data in the data processing apparatus 100, according to an embodiment.

Referring to FIG. 6, the data processing apparatus 100 may output a user interface 600 for allowing alignment of scan data with the occlusion surface to determine the direction of base generation.

In the user interface 600 output as such, when the user selects a plurality of points, for example, three or four points, in scan data 500, the data processing apparatus 100 may align the occlusion surface based on the plurality of selected points. Here, the lingual side and the buccal side in the scan data 500 may be divided. The occlusion axis of the scan data may be automatically calculated.

Returning back to FIG. 4, in operation 420, the data processing apparatus 100 may generate a border of a base based on the scan data.

The base may refer to a support structure connected to each of an upper-jaw portion and a lower-jaw portion for combining the patient's upper jaw and lower jaw to view the occlusion relationship between the patient's upper-jaw teeth and lower-jaw teeth. The base may include an upper-jaw base connected to the upper-jaw portion and a lower-jaw base connected to the lower-jaw portion.

FIG. 7 is a reference diagram for describing an example of a method of generating a border of an upper-jaw base, according to an embodiment.

Referring to FIG. 7, the data processing apparatus 100 may calculate a bounding box 750 that is perpendicular to the occlusion axis of the scan data 500 and surrounds the boundary of the scan data, and generate a border of the base extending by a certain distance from the bounding box 750. As an example, two vertexes 701 and 702 of the base may be generated by vertically descending by a certain distance d from the vertexes at both ends of the bottom side of the bounding box 750. Here, the bottom side of the bounding box 750 may refer to a surface generated in the lingual direction of the scan data. Based on the two vertexes 701 and 702 generated as such, other vertexes 703 to 707 may be obtained according to a predetermined angle. Also, a border of the base may be obtained according to the vertexes 701 to 707.

In the example illustrated in FIG. 7, because an ABO base is used as an example, the description is based on a heptagonal base; however, this is merely an example. Even when the base has a different shape, when the bounding box may be generated based on the scan data and the base may be generated based on the bounding box, it may be understood that the base may have any polygonal shape.

Also, although the upper-jaw base is illustrated as an example in FIG. 7, the lower-jaw base may also be generated in the same way.

In operation 430, the data processing apparatus 100 may obtain a gingiva area from the scan data.

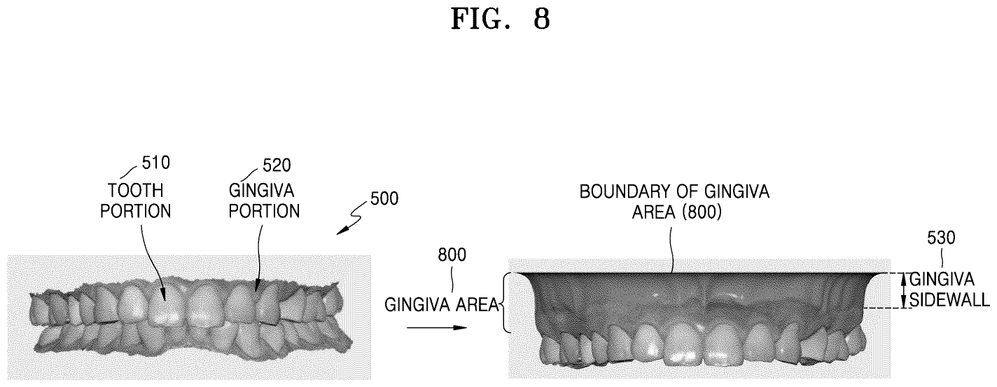

FIG. 8 is a reference diagram for describing a method of obtaining a gingiva area from scan data.

According to an example, the data processing apparatus 100 may trim an edge portion in the gingiva portion 520 of the scan data 500 and generate a virtual sidewall 530 connected to the gingiva portion 520. Because the scan data includes vertexes, a virtual gingiva sidewall 530 may be generated by generating vertexes constituting the sidewall to be connected to the gingiva portion 520. The data processing apparatus 100 may obtain a gingiva area 800 by extending the gingiva sidewall 530 to the gingiva portion 520 of the scan data 500.

The height of the gingiva sidewall 530 to be generated may vary depending on the height of the gingiva portion 520 included in the scan data 500. For example, when the scan data 500 includes only the gingiva portion 520 with a very low height, the height of the gingiva sidewall 530 may be relatively high. For example, when the scan data 500 includes the gingiva portion 520 with a very high height, the height of the gingiva sidewall 530 may be relatively low.

Herein, the term “gingiva area” is used to represent a portion connected to the border of the base, which may be distinguished from the gingiva portion 520 included in the scan data obtained by initially scanning the oral cavity.

In operation 440, the data processing apparatus 100 may obtain tooth model data by generating a mesh between the gingiva area and the border of the base (inside the border of the base). In this case, the data processing apparatus 100 may calculate and generate the position of an auxiliary line inside the border of the base (the position corresponding to the palate), generate a mesh by extending a gingiva area (a lingual area) corresponding to the palate to the auxiliary line, and obtain tooth model data by generating a mesh inside the auxiliary line.

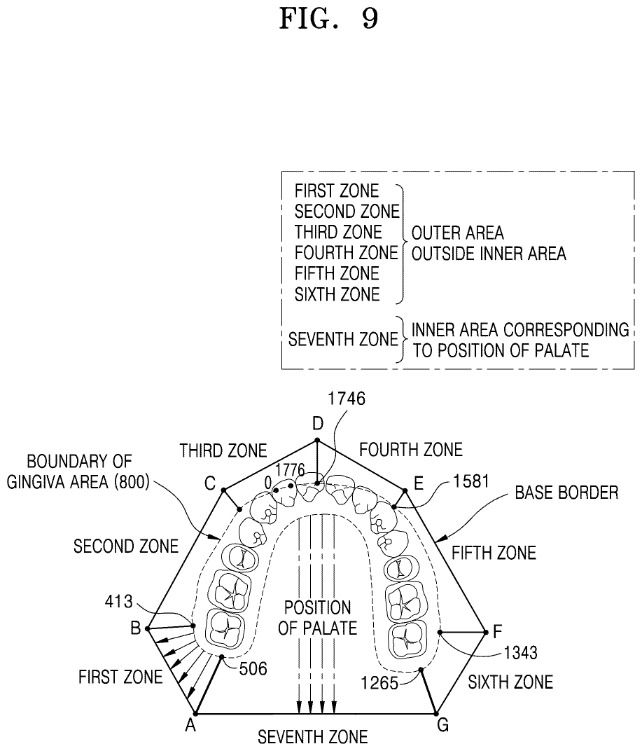

Hereinafter, a method of generating a mesh between the gingiva area and the border of the base will be described with reference to FIGS. 9 to 11.

FIG. 9 is a reference diagram for describing a method of dividing an area between the gingiva area and the base into a plurality of zones and generating a mesh in an outer area, according to an embodiment.

Referring to FIG. 9, the data processing apparatus 100 may divide an area between the gingiva area 800 and the base border into a plurality of zones. Particularly, the data processing apparatus 100 may divide the area into a plurality of zones by connecting the vertexes at the boundary of the gingiva area 800 at the shortest distance from each vertex of an N-polygonal shape constituting the base border. Referring to FIG. 8, the boundary of the gingiva area 800 may represent the most edge portion of the gingiva area including the gingiva sidewall and may represent a portion indicated by a dotted line. For example, as illustrated in FIG. 9, when a figure constituting the base border is a heptagon, it may be divided into seven zones by connecting the vertexes of the gingiva area 800 at the shortest distance from each of seven vertexes. Referring to FIG. 9, the vertex of the boundary of the gingiva area 800 at the shortest distance from a vertex A of the base border may be a vertex 506, the vertex of the boundary of the gingiva area 800 at the shortest distance from a vertex B may be a vertex 413, the vertex of the boundary of the gingiva area 800 at the shortest distance from a vertex C may be a vertex 110, the vertex of the boundary of the gingiva area 800 at the shortest distance from a vertex D may be a vertex 1746, the vertex of the boundary of 800 at the shortest distance from a vertex E may be a vertex 1581, the vertex of the boundary of 800 at the shortest distance from a vertex F may be a vertex 1343, and the vertex of the boundary of 800 at the shortest distance from a vertex G may be a vertex 1265.

The zone between the vertexes A and B may be defined as a first zone, the zone between the vertexes B and C may be defined as a second zone, the zone between the vertexes C and D may be defined as a third zone, the zone between the vertexes D and E may be defined as a fourth zone, the zone between the vertexes E and F may be defined as a fifth zone, the zone between the vertexes F and G may be defined as a sixth zone, and the zone between the vertexes G and A may be defined as a seventh zone. Also, based on the gingiva area 800, the seventh zone that is the zone corresponding to the palate position may be represented as an inner area, and the first to sixth areas that are areas outside the inner area may be represented as an outer area. The plurality of zones may be divided into the outer area and the inner area based on the area corresponding to the palate position because a method of generating a mesh may vary due to the difference in the shape of the gingiva area in each zone. Because the gingiva area from the first to sixth zones corresponding to the outer area is convex and the distance from the boundary of the gingiva area to the base border is relatively short, a mesh may be generated by simply connecting the points from the boundary of the gingiva area to the base border. On the other hand, in the seventh zone corresponding to the inner area, because the gingiva area is concave, when a mesh is generated by connecting the points on a straight base border, wrinkles may occur therein and thus the shape of the generated mesh may be unnatural. Thus, as for the seventh zone corresponding to the inner area, another method other than simple point connection may be required to naturally generate a mesh.

As for the first to sixth zones corresponding to the outer area, a mesh may be generated by connecting each vertex of the boundary of the gingiva area 800 and the point of the base border, and in this case, a curve may be made to generate a mesh such that the gingiva edge may be smoothly connected to the base border. The boundary of the gingiva area 800 may include a plurality of vertexes. For example, in FIG. 9, it may be seen that the boundary of the gingiva area 800 includes a total of 1777 vertexes including vertexes 0 to 1776 in the third zone.

For example, the first zone will be described as an example. The boundary of the gingiva area 800 corresponding to the first zone may include vertexes 413 to 506. Because the vertex 506 corresponds to a point A and the vertex 413 corresponds to a point B, the data processing apparatus 100 may generate, between the base borders A and B, a point corresponding to each vertex of the vertexes 414 to 505 that are the vertexes between the vertex 506 and the vertex 413. For example, 92 points may be generated at uniform intervals between the point A and the point B. Also, the data processing apparatus 100 may connect each vertex existing at the boundary of the gingiva area 800 with the corresponding point at the base border and generate a mesh based on the point connection. As for the second to sixth zones that are the other zones of the outer area, a mesh may also be generated according to the mesh generation method in the first zone.

However, as for the seventh zone corresponding to the inner area, when a mesh is generated by simply connecting the vertexes at the boundary of the gingiva area and the points of the base border, that is, as the gingiva area boundary of the front-tooth portion has a concave shape, when a mesh is generated by connecting the vertexes at the boundary of the gingiva area of the front-tooth portion to the points of the straight base border corresponding to the seventh zone, the mesh may be wrinkled in a portion corresponding to the palate position and thus may be generated to be very unnatural. Thus, it may be necessary to generate a mesh in a different way for the seventh zone corresponding to the inner area.

For this purpose, the data processing apparatus 100 may divide an area corresponding to the seventh zone into a plurality of subareas such that a natural mesh may be generated in the area corresponding to the seventh zone. Also, the data processing apparatus 100 may use an auxiliary line as a reference for dividing the area corresponding to the seventh zone into a plurality of subareas. The auxiliary lines may have a curved or parabolic shape. The auxiliary line may be generated according to various known curve generation techniques; for example, a Bezier curve may be used.

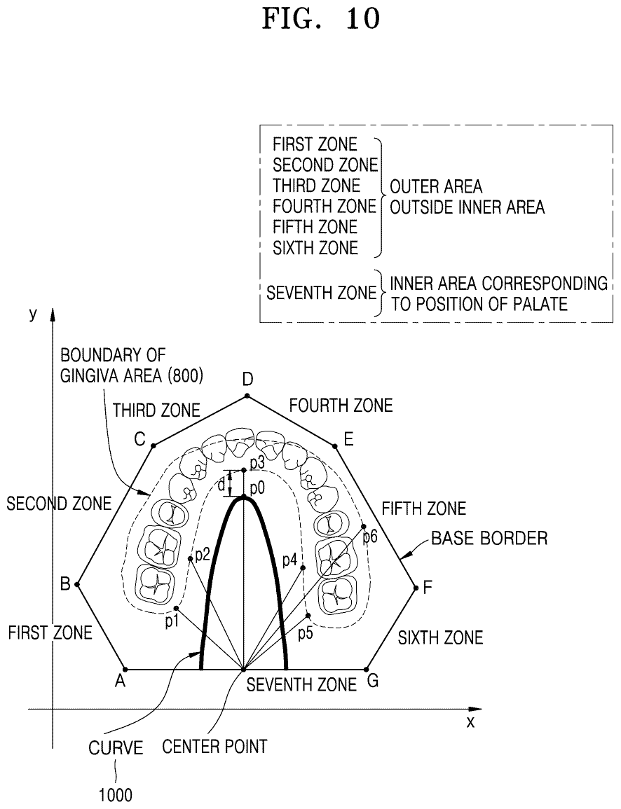

FIG. 10 is a reference diagram for describing a method of generating a curve in an area corresponding to the position of a palate, according to an embodiment.

Referring to FIG. 10, coordinates may be obtained by projecting the scan data onto the xy coordinate plane. Next, a center point of a portion corresponding to the seventh zone of the base border, that is, a section from A to G may be found, a line may be drawn from the center point to the boundary point of the scan data, and the most distant vertex may be found among the distances from the center point to the boundary point of the scan data. For example, referring to FIG. 10, a vertex p3 corresponding to the farthest distance among the distances from the center point to the boundary point of the scan data may be found in the same way from the center point to p1, from the center point to p2, from the center point to p3, from the center point to p4, from the center point to p5, and from the center point to p6. In this case, in order to exclude the buccal side of the scan data, a case where there is a point intersecting a line as in p6 may be excluded.

As such, the vertex p3 corresponding to the farthest distance may be found, and the found vertex p3 or a point within a certain distance d from the found vertex p3 may be set as a reference point p0. Also, a curve connecting from the reference point p0 to a portion corresponding to the seventh zone of the base border (in the lingual direction) may be generated. In this case, a Bezier curve may be used as the curve.

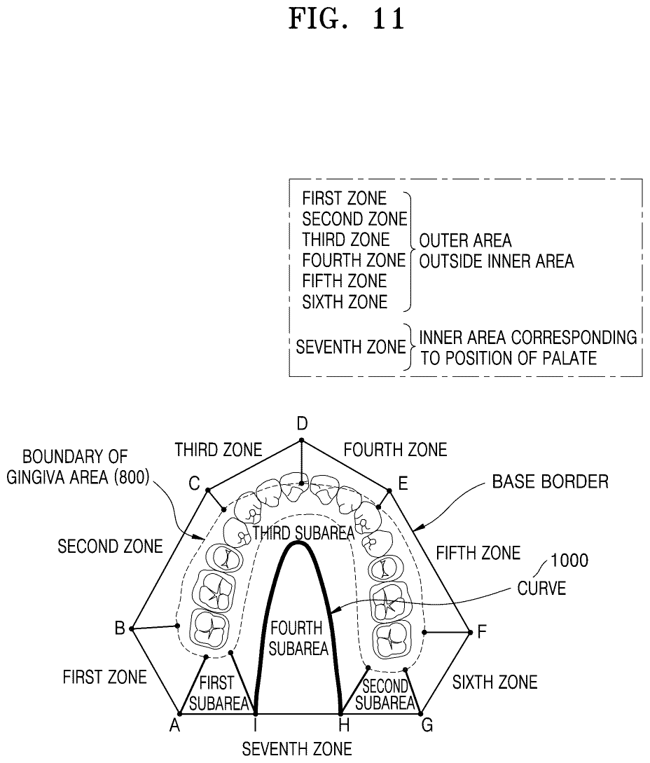

FIG. 11 is a reference diagram for describing a method of dividing an area between the gingiva area and the base into a plurality of zones and generating a mesh in an inner area, according to an embodiment.

Referring to FIG. 11, the data processing apparatus 100 may divide an area corresponding to the seventh zone into a plurality of subareas such that a natural mesh may be generated in the area corresponding to the seventh zone. For this purpose, the data processing apparatus 100 may generate a curve 1000 in an area corresponding to the palate position, as described in FIG. 10.

Also, the data processing apparatus 100 may identify vertexes at the boundary of the gingiva area located at the shortest distance from points H and I at which the curve 1000 meets the base border. For example, the vertex at the boundary of the gingiva area located at the shortest distance from the point H is represented as a vertex 1100, and the vertex at the boundary of the gingiva area located at the shortest distance from the point I is represented as a vertex 610. For convenience, in the seventh zone, an area including points A to I and vertexes 506 to 610 may be defined as a first subarea, an area including points H to G and vertexes 1100 to 1265 may be defined as a second subarea, an area between the boundary of the gingiva area and the curve 1000 may be defined as a third subarea, and an inner area of the curve 1000 may be defined as a fourth subarea.

Also, the data processing apparatus 100 may generate a mesh in units of each subarea constituting the seventh zone.

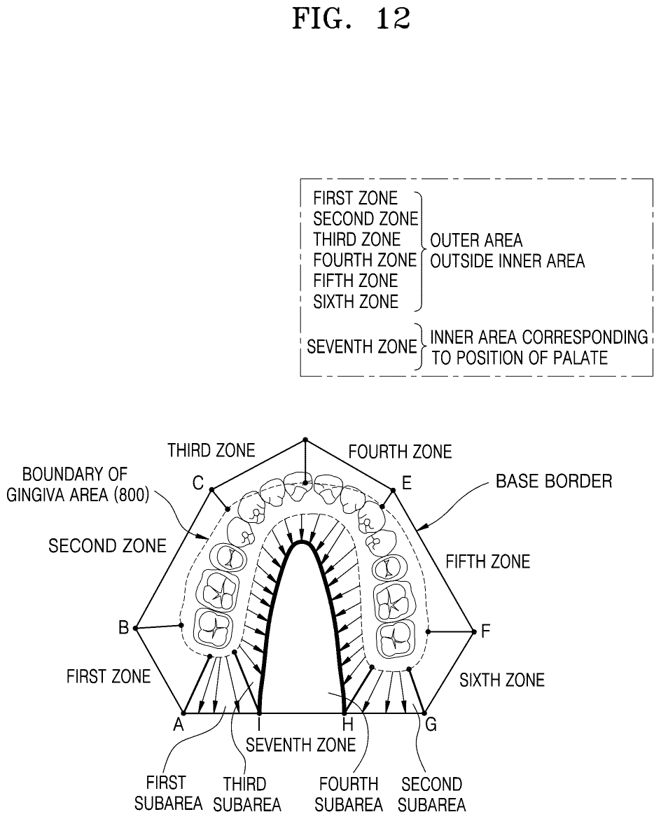

FIG. 12 is a reference diagram for describing a method of generating a mesh for each subarea in a seventh zone that is an inner area, according to an embodiment.

Referring to FIG. 12, the data processing apparatus 100 may generate a mesh for the first subarea and the second subarea in the same way as the method of generating a mesh in the first to sixth areas corresponding to the outer area. That is, the data processing apparatus 100 may generate points in the section between the point A and the point I of the base border as many as the number of vertexes included in the boundary of the gingiva area corresponding to the first subarea, connect the vertexes at the boundary of the gingiva area to the points generated at the base border, and generate a mesh based on the point connection. The data processing apparatus 100 may generate a mesh for the second subarea in the same way.

Next, for the third subarea, the data processing apparatus 100 may generate points in the section of the curve 1000 as many as the number of vertexes included in the boundary of the gingiva area corresponding to the third subarea, connect the points at the boundary of the gingiva area corresponding to the third subarea to the points generated at the curve 1000, and generate a mesh based on the point connection.

Next, the data processing apparatus 100 may generate a certain mesh in the fourth subarea, that is, an area including the section between the curve 1000 and the point H and the point I of the base border.

As such, the data processing apparatus 100 may prepare a curve at a position corresponding to the palate and generate a mesh based on the prepared curve to generate a base with a smoother and softer curve without wrinkling in an area corresponding to the palate.

An intraoral image processing method according to an embodiment of the present disclosure may be embodied in the form of program commands executable through various computer means and then may be recorded on a computer-readable recording medium. Also, an embodiment of the present disclosure may be a computer-readable storage medium having recorded thereon one or more programs including at least one instruction for executing the intraoral image processing method.

The computer-readable storage medium may include program instructions, data files, and data structures either alone or in combination. Here, examples of the computer-readable storage medium may include magnetic media such as hard disks, floppy disks, and magnetic tapes, optical media such as CD-ROMs and DVDs, magneto-optical media such as floptical disks, and hardware devices such as ROMs, RAMs, and flash memories that are configured to store and execute program instructions.

Here, a machine-readable storage medium may be provided in the form of a non-transitory storage medium. Here, the ‘non-transitory storage medium’ may mean that the storage medium is a tangible device. Also, the ‘non-transitory storage medium’ may include a buffer in which data is temporarily stored.

According to an embodiment, the intraoral image processing method according to various embodiments described herein may be included and provided in a computer program product. The computer program product may be distributed in the form of a machine-readable storage medium (e.g., a compact disc read only memory (CD-ROM)). Alternatively, the computer program product may be distributed (e.g., downloaded or uploaded) online through an application store (e.g., Play Store) or directly between two user devices (e.g., smart phones). Particularly, the computer program product according to the described embodiments may include a storage medium having recorded thereon a program including at least one instruction for performing the intraoral image processing method according to the described embodiments.

Although embodiments have been described above in detail, the scope of the present disclosure is not limited thereto and various modifications and improvements made by those of ordinary skill in the art by using the basic concept of the present disclosure defined in the following claims are also included in the scope of the present disclosure.

1. A method of processing a three-dimensional intraoral model, the method comprising:
obtaining scan data by scanning an object;
generating a border of a base based on the scan data;
obtaining a gingiva area from the scan data; and
obtaining tooth model data by generating a mesh between the gingiva area and the border of the base and connecting the gingiva area and the border of the base by using the mesh, another mesh different from the mesh being generated at a position corresponding to a palate.

2. The method of claim 1, wherein the obtaining of the tooth model data comprises generating the another mesh at the position corresponding to the palate by using an auxiliary line.

3. The method of claim 2, wherein the obtaining of the tooth model data comprises generating the another mesh by generating the auxiliary line at the position corresponding to the palate in the gingiva area and extending boundary points of the gingiva area corresponding to the palate to the auxiliary line.

4. The method of claim 3, wherein the obtaining of the tooth model data comprises:
mapping the boundary points of the gingiva area corresponding to the palate to points of the auxiliary line and generating a mesh based on the mapped points; and
generating a mesh inside the auxiliary line.

5. The method of claim 2, wherein the auxiliary line includes a curve or a parabola.

6. The method of claim 2, wherein the obtaining of the tooth model data comprises:
dividing a boundary of the gingiva area into a plurality of boundary zones to correspond to sides of a polygon constituting the base;
generating points at a side of the base corresponding to each boundary zone to correspond to points of each boundary zone of the gingiva area; and
generating the another mesh by mapping the points of each boundary zone of the gingiva area to the points of the side corresponding to each boundary zone.

7. The method of claim 6, wherein the obtaining of the tooth model data comprises:
with respect to a zone corresponding to the palate, mapping at least some of points of a boundary zone of the gingiva area to points of the auxiliary line and generating a mesh based on the mapped points; and
generating a mesh inside the auxiliary line.

8. The method of claim 2, wherein the obtaining of the gingiva area comprises identifying a tooth portion and a gingiva portion from the scan data and obtaining the gingiva area by generating a gingiva sidewall by extending the gingiva portion.

9. A data processing apparatus for processing a three-dimensional intraoral model, the data processing apparatus comprising:
a memory storing one or more instructions; and
a processor configured to execute the one or more instructions stored in the memory to:
obtain scan data by scanning an object;
generate a border of a base based on the scan data;
obtain a gingiva area from the scan data; and
obtain tooth model data by generating a mesh between the gingiva area and the border of the base and connecting the gingiva area and the border of the base by using the mesh, another mesh different from the mesh being generated at a position corresponding to a palate.

10. The data processing apparatus of claim 9, wherein the processor is further configured to execute the one or more instructions to, in order to obtain the tooth model data, generate the another mesh at the position corresponding to the palate by using an auxiliary line.

11. The data processing apparatus of claim 10, wherein the processor is further configured to execute the one or more instructions to, in order to obtain the tooth model data, generate the another mesh by generating the auxiliary line at the position corresponding to the palate in the gingiva area and extending boundary points of the gingiva area corresponding to the palate to the auxiliary line.

12. The data processing apparatus of claim 11, wherein the processor is further configured to execute the one or more instructions to, in order to obtain the tooth model data:
map the boundary points of the gingiva area corresponding to the palate to points of the auxiliary line and generating a mesh based on the mapped points; and
generate a mesh inside the auxiliary line.

13. The data processing apparatus of claim 10, wherein the auxiliary line includes a curve or a parabola.

14. The data processing apparatus of claim 10, wherein the processor is further configured to execute the one or more instructions to, in order to obtain the tooth model data:
divide a boundary of the gingiva area into a plurality of boundary zones to correspond to sides of a polygon constituting the base;
generate points at a side of the base corresponding to each boundary zone to correspond to points of each boundary zone of the gingiva area; and
generate the another mesh by mapping the points of each boundary zone of the gingiva area to the points of the side corresponding to each boundary zone.

15. The data processing apparatus of claim 13, wherein the processor is further configured to execute the one or more instructions to, in order to obtain the tooth model data:
with respect to a zone corresponding to the palate, map at least some of points of a boundary zone of the gingiva area to points of the auxiliary line and generate a mesh based on the mapped points; and
generate a mesh inside the auxiliary line.

16. The data processing apparatus of claim 10, wherein the processor is further configured to execute the one or more instructions to, in order to obtain the gingiva area, obtain the gingiva area by identifying a tooth portion and a gingiva portion from the scan data and generating a gingiva sidewall by extending the gingiva portion.

17. A computer-readable recording medium having recorded thereon a program for performing, by a data processing apparatus, a method of processing a three-dimensional intraoral model, the method comprising:
obtaining scan data by scanning an oral cavity;
generating a border of a base based on the scan data;
obtaining a gingiva area from the scan data; and
obtaining tooth model data by generating a mesh between the gingiva area and the border of the base and connecting the gingiva area and the border of the base by using the mesh, another mesh different from the mesh being generated at a position corresponding to a palate.
